(19) United States
(12) Patent Application Publication
(10) Pub. No.: US 2023/0024202 A1
(43) Pub. Date: Jan. 26, 2023

(54) METHOD FOR ANALYZING DEGARELIX AND ASSOCIATED PRODUCTS

(71) Applicant: FRESENIUS KABI USA, LLC, Lake Zurich, IL (US)

(72) Inventors: Jagdish LANDE, Lake Zurich, IL (US); Kamlesh PATEL, Lake Zurich, IL (US); Umer RAHEEMI, Lake Zurich, IL (US)

(73) Assignee: FRESENIUS KABI USA, LLC, Lake Zurich, IL (US)

(21) Appl. No.: 17/782,422

(22) PCT Filed: Dec. 3, 2020
(86) PCT No.: PCT/US2020/063114

§ 371 Date: Jun. 3, 2022

Related U.S. Application Data
(60) Provisional application No. 62/944,276, filed on Dec. 5, 2019.

(51) Int. Cl. C07K 1/16; G01N 33/76; C07K 14/59; B01J 20/26; B01D 15/42

(52) U.S. Cl. CPC C07K 1/16; G01N 33/76; C07K 14/59; B01J 20/265; B01D 15/426; A61K 38/00

(57) ABSTRACT

Provided are methods for analyzing and purifying degarelix or a pharmaceutically acceptable salt thereof containing at least one related impurity. Also provided are methods for analyzing and purifying degarelix or a pharmaceutically acceptable salt thereof containing compound A and/or compound D as an impurity. Further provided are degarelix or a pharmaceutically acceptable salt thereof prepared from and/or selected using the disclosed methods.

### CROSS-REFERENCE TO RELATED APPLICATIONS

This patent application claims the benefit of U.S. Provisional Application No. 62/944,276, filed Dec. 5, 2019, the disclosure of which is incorporated herein by reference in its entirety for all purposes.

### BACKGROUND OF THE INVENTION

Degarelix (e.g., degarelix acetate) is a gonadotropin-releasing hormone (GnRH) used as a hormonal therapy in the treatment of prostate cancer. Degarelix (e.g., degarelix acetate) has an immediate onset of action and suppresses gonadotropins, testosterone, and prostate-specific antigen (PSA). Degarelix (e.g., degarelix acetate) is a synthetic peptide commonly marketed as the acetate, N-acetyl-3-(2-naphthyl)-D-alanyl-4-chloro-D-phenylalanyl-3-(3-pyridyl)-D-alanyl-L-seryl-4-((S)-dihydroorotamido)-L-phenylalanyl-4-ureido-D-phenylalanyl-L-leucyl-N6-isopropyl-L-lysyl-L-prolyl-D-alaninamide.

Conventional synthesis techniques for the production of degarelix (e.g., degarelix acetate) can result in certain impurities (e.g., compounds A-G, I, K, and M) which can be challenging to analyze and/or separate from degarelix (e.g., degarelix acetate). In particular, compounds A and D can not only be difficult to separate from degarelix, but also from each other making it difficult to quantify the individual impurities.

In view of the foregoing, improved methods are needed to analyze each of the impurities commonly present in degarelix (e.g., degarelix acetate), as well as to provide preparatory techniques to separate impurities, e.g., compounds A-G, I, K, and M, and more particularly compounds A and D, from degarelix (e.g., degarelix acetate).

### BRIEF SUMMARY OF THE INVENTION

The invention provides a method for analyzing a sample comprising degarelix or a pharmaceutically acceptable salt thereof and at least one related impurity. In one respect, the method comprises:
(a) eluting the sample through a high pressure liquid chromatography (HPLC) column to produce a chromatogram that resolves the degarelix or pharmaceutically acceptable salt thereof and the at least one related impurity, wherein the chromatogram comprises a first peak with a first area representing degarelix in the sample, and a second peak with a second area representing the at least one related impurity in the sample;
(b) determining the first area under the first peak representing the degarelix or pharmaceutically acceptable salt thereof in the sample,
(c) determining the second area under the second peak representing the at least one related impurity in the sample, and
(d) determining the concentration of the at least one related impurity in the sample based on the first and second areas. The eluting comprises eluting the sample with a mobile phase A comprising a first aqueous solution having a first pH and a first organic solvent, and with a mobile phase B comprising a second aqueous solution having a second pH, and a second organic solvent, wherein the first and the second pH values are different. The invention further provides for the selection of a batch of degarelix or a pharmaceutically acceptable salt thereof based on the results of this method, and a batch selected thereby.

The invention also provides a method for analyzing a sample comprising degarelix or a pharmaceutically acceptable salt thereof and compound A, which method includes:
(a) eluting the sample isocratically with a mobile phase through a high performance liquid chromatography (HPLC) column to produce a chromatogram that resolves the degarelix and compound A, wherein the chromatogram comprises a first peak with a first area representing degarelix in the sample, and a second peak with a second area representing compound A in the sample;
(b) determining the first area under the first peak representing the degarelix or pharmaceutically acceptable salt thereof in the sample,
(c) determining the second area under the second peak representing the compound A in the sample, and
(d) determining the concentration of the compound A in the sample based on the first and second areas. The invention further provides for the selection of a batch of degarelix or a pharmaceutically acceptable salt thereof based on the results of this method, and a batch selected thereby.

The invention also provides a method for analyzing a sample comprising degarelix or a pharmaceutically acceptable salt thereof, compound A, and at least one related impurity other than compound A, which method includes:
(a) eluting a first portion of the sample through a high performance liquid chromatography (HPLC) column to produce a first chromatogram that resolves the degarelix or pharmaceutically acceptable salt thereof and the at least one related impurity, the first chromatogram comprising a first peak with a first area representing degarelix in the sample, and a second peak with a second area representing the at least one related impurity in the sample, wherein the eluting of the first portion of sample comprises eluting with a mobile phase A comprising a first aqueous solution having a first pH and a first organic solvent, and with a mobile phase B comprising a second aqueous solution having a second pH and a second organic solvent, wherein the first and the second pH values are different, and
(b) eluting a second portion of the sample through a HPLC column, to produce a second chromatogram that resolves the degarelix and compound A, the second chromatogram comprising a first peak with a first area representing degarelix in the sample, and a second peak with a second area representing compound A in the sample, wherein the eluting of the second portion of sample comprises isocratically eluting with a mobile phase,
(c) determining the first areas under the first peaks representing the degarelix or pharmaceutically acceptable salt thereof in the first and second chromatograms,
(d) determining the second area under the peak representing the at least one related impurity in the first chromatogram,
(e) determining the second area under the second peak representing the compound A in the second chromatogram, and determining the concentrations of the at least one related impurity and the compound A in the sample based on the first and second areas of the first and second chromatograms. The invention further provides for the selection of a batch of degarelix or a pharmaceutically acceptable salt thereof based on the results of this method, and a batch selected thereby.

The invention also provides a method of purifying degarelix or a pharmaceutically acceptable salt thereof and compound A, which method comprises:
(a) eluting the degarelix through a chromatographic column with a mobile phase to separate the degarelix or pharmaceutically acceptable salt thereof containing compound A, to produce a purified form of degarelix or a pharmaceutically acceptable salt thereof, and
(b) isolating the purified degarelix or pharmaceutically acceptable salt thereof, wherein the eluting comprises isocratically eluting the degarelix from the chromatographic column with the mobile phase. The invention further provides a purified form of degarelix or a pharmaceutically acceptable salt thereof prepared by this and other methods described herein.

### BRIEF DESCRIPTION OF THE DRAWINGS

FIG. 1 illustrates a high performance liquid chromatography chromatogram of a resolution solution analyzed according to an exemplary method.

FIG. 2 illustrates a high performance liquid chromatography chromatogram of a synthetic sample analyzed according to an exemplary method.

FIG. 3 illustrates a high performance liquid chromatography chromatogram of a resolution solution prepared and analyzed according to an exemplary method.

FIG. 4 illustrates a high performance liquid chromatography chromatogram of a synthetic sample analyzed according to an exemplary method.

### DETAILED DESCRIPTION OF THE INVENTION

The invention provides methods for analyzing, separating, and/or purifying degarelix or a pharmaceutically acceptable salt thereof (e.g., degarelix acetate) containing certain impurities (e.g., certain impurities identified as compounds A-G, I, K, and M). As used herein, the term “degarelix” refers to N-acetyl-3-(2-naphthyl)-D-alanyl-4-chloro-D-phenylalanyl-3-(3-pyridyl)-D-alanyl-L-seryl-4-((S)-dihydroorotamido)-L-phenylalanyl-4-ureido-D-phenylalanyl-L-leucyl-N6-isopropyl-L-lysyl-L-prolyl-D-alaninamide. The degarelix to be analyzed and/or purified according to the invention can be in its free-base form or can exist as a pharmaceutically acceptable salt thereof. In certain embodiments, the degarelix described herein is in the form of degarelix acetate.

As used herein, the phrase “pharmaceutically acceptable salt” is intended to include salts derived from the parent compound which contains a basic or acidic moiety. Generally, such salts can be prepared by reacting the free acid or base forms of these compounds with a stoichiometric amount of the appropriate base or acid, respectively, in water or in an organic solvent, or in a mixture of the two. For example, an inorganic acid (e.g., hydrochloric acid, sulfuric acid, phosphoric acid, or hydrobromic acid), an organic acid (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base (e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typically utilized. Lists of suitable salts are found in Remington's Pharmaceutical Sciences, 18th ed., Mack Publishing Company, Easton, Pa., 1990, p. 1445, and Journal of Pharmaceutical Science, 66: 2-19 (1977).

Impurities of degarelix may be synthetic intermediates, synthetic byproducts, degradation products, or aggregation products. For example, impurities related to degarelix may include impurities identified as compounds A-G, I, K, and M, as described herein, the structures for which are provided in Table 1.

TABLE 1
Degarelix Impurities

| Com- | |
|---|---|
| pound | Structure (Chemical Name) |
| A | 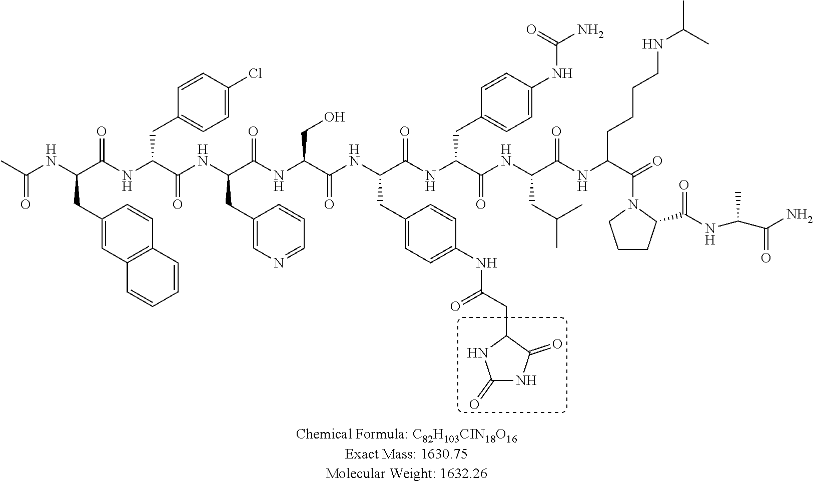 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)CC2NC(=O)NC2=O)cc1)C(=O)N[C@H](Cc1ccc(NC(N)=O)cc1)C(=O)N[C@@H](CC(C)C)C(=O)NC(CCCCNC(C)C)C(=O)N1CCC[C@H]1C(=O)N[C@H](C)C(N)=O] |
| | (D-alaninamide, N-acetyl-3-(2-naphthalenyl)-D-alanyl-4-chloro-D-phenylalanyl-3-(3-pyridyl)-D-alanyl-L-seryl-4-([2-(5-hydantoyl)]- |
| | acetylamino-L-phenylalanyl-4-[(aminocarbonyl)amino]-D-phenylalanyl-L-leucyl-N6-(1-methylethyl)-L-lysyl-L-prolyl) |
| B | 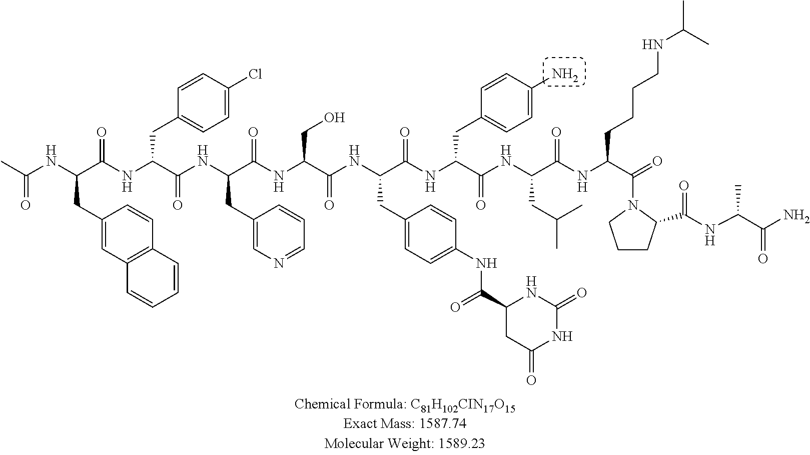 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)[C@@H]2CC(=O)NC(=O)N2)cc1)C(=O)N[C@H](Cc1ccc(N)cc1)C(=O)N[C@@H](CC(C)C)C(=O)N[C@@H](CCCCNC(C)C)C(=O)N1CCC[C@H]1C(=O)N[C@H](C)C(N)=O] |
| | (D-alaninamide, N-acetyl-3-(2-naphthalenyl)-D-alanyl-4-chloro-D-phenylalanyl-3-(3-pyridyl)-D-alanyl-L-seryl-4-[[[(4S)-hexahydro- |
| | 2,6-dioxo-4-pyrimidinyl]carbonyl]amino]-L-phenylalanyl-4-amino-D-phenylalanyl-L-leucyl-N6-(1-methylethyl)-L-lysyl-L-prolyl) |
| C | 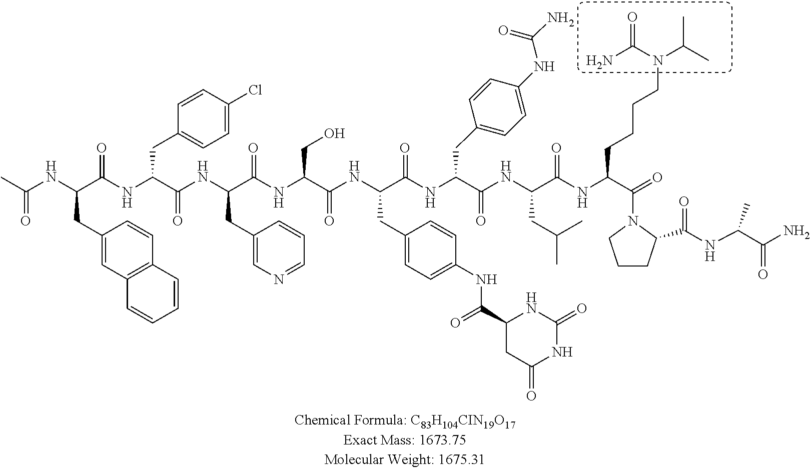 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCN(C(N)=O)C(C)C)C(=O)N1CCC[C@H]1C(=O)N[C@H](C)C(N)=O] |
| | (D-alaninamide, N-acetyl-3-(2-naphthalenyl)-D-alanyl-4-chloro-D-phenylalanyl-3-(3-pyridyl)-D-alanyl-L-seryl-4-[[[(4S)-hexahydro- |
| | 2,6-dioxo-4-pyrimidinyl]carbonyl]amino]-L-phenylalanyl-4-[(aminocarbonyl)amino]-D- |
| | phenylalanyl-L-leucyl-N6-(1-methylethyl,aminocarbonyl)-L-lysyl-L-prolyl) |
| D | 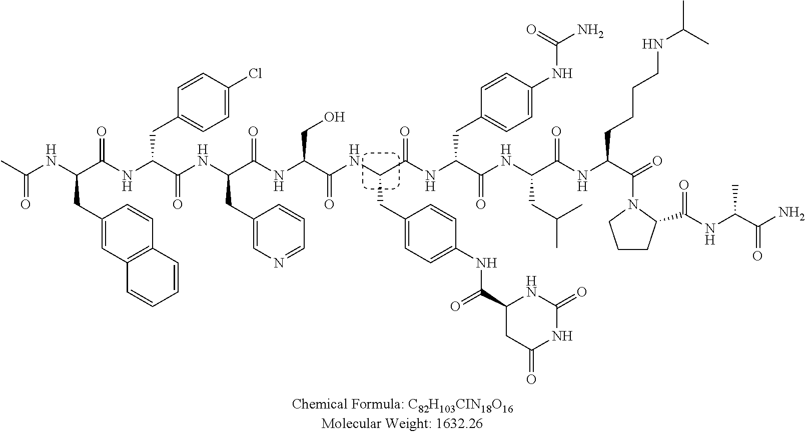 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@H](C)C(N)=O] |
| | (D-alaninamide, N-acetyl-3-(2-naphthalenyl)-D-alanyl-4-chloro-D-phenylalanyl-3-(3-pyridyl)-D-alanyl-L-seryl-4-[[[(4S)-hexahydro- |
| | 2,6-dioxo-4-pyrimidinyl]carbonyl]amino]-D-phenylalanyl-4-[(aminocarbonyl)amino]-D- |
| | phenylalanyl-L-leucyl-N6-(1-methylethyl)-L-lysyl-L-prolyl) |
| E | 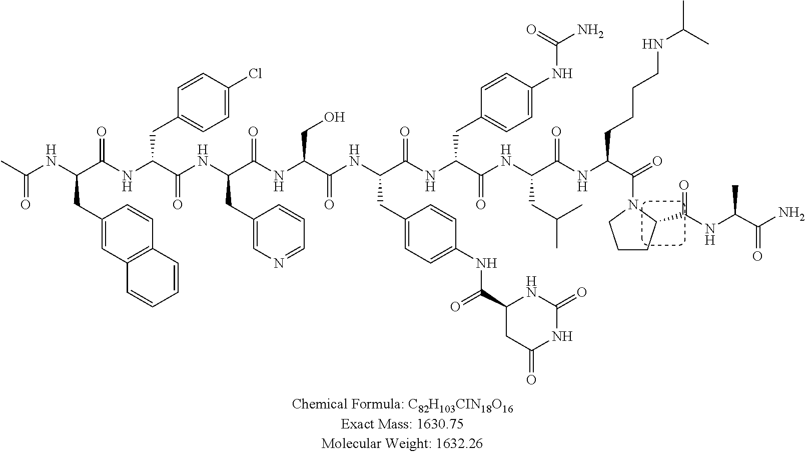 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@@H](C)C(N)=O] |
| | (Alaninamide, N-acetyl-3-(2-naphthalenyl)-D-alanyl-4-chloro- D-phenylalanyl-3-(3-pyridyl)-D-alanyl-L-seryl-4-[[[(4S)-hexahydro- |
| | 2,6-dioxo-4-pyrimidinyl]carbonyl]amino]-D-phenylalanyl-4-[(aminocarbonyl)amino]-D- |
| | phenylalanyl-L-leucyl-N6-(1-methylethyl)-L-lysyl-Lprolyl) |
| F | 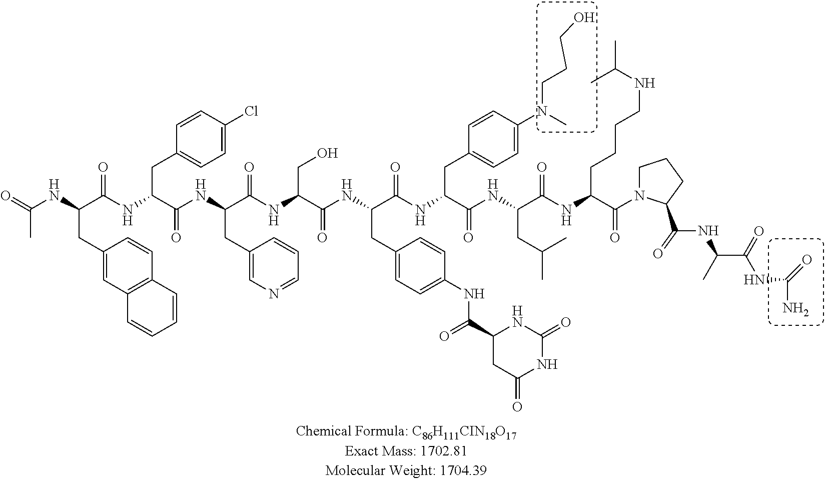 |
| | (D-alaninamide Carboamide, N-acetyl-3-(2-naphthalenyl)-D-alanyl-4-chloro-D-phenylalanyl-3-(3-pyridyl)-D-alanyl-L-seryl-4-[[[(4S)- |
| | hexahydro-2,6-dioxo-4-pyrimidinyl]carbonyl]amino]-D-phenylalanyl-4-[(3- |
| | hydroxypropyl)(methyl)amino]-D-phenylalanyl-L-leucyl-N6-(1-methylethyl)-L-lysyl-L-prolyl) |
| G | 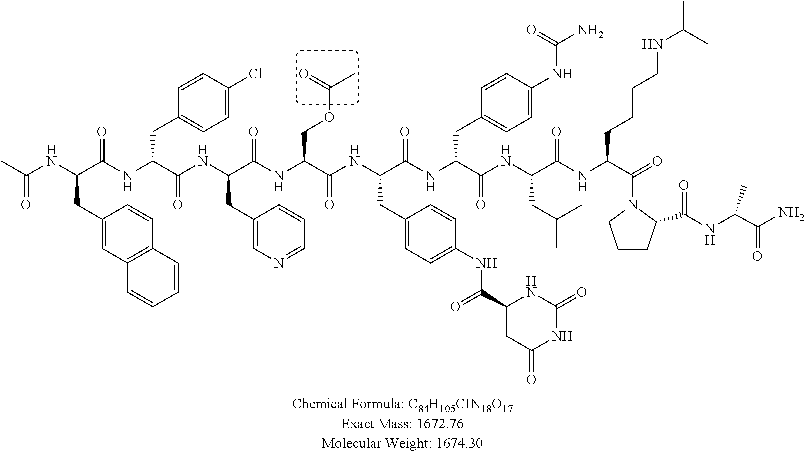 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](COC(C)=O)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@H](C)C(N)=O] |
| | (D-alaninamide, N-acetyl-3-(2- naphthalenyl)-D-alanyl-4-chloro-D-phenylalanyl-3-(3-pyridyl)-D-alanyl-O3-(acetyl)-L-seryl-4-[[[(4S)- |
| | hexahydro-2,6-dioxo-4-pyrimidinyl]carbonyl]amino]-D-phenylalanyl-4- |
| | [(aminocarbonyl)amino]-D-phenylalanyl-L-leucyl-N6-(1-methylethyl)-L-lysyl-L-prolyl) |
| I | 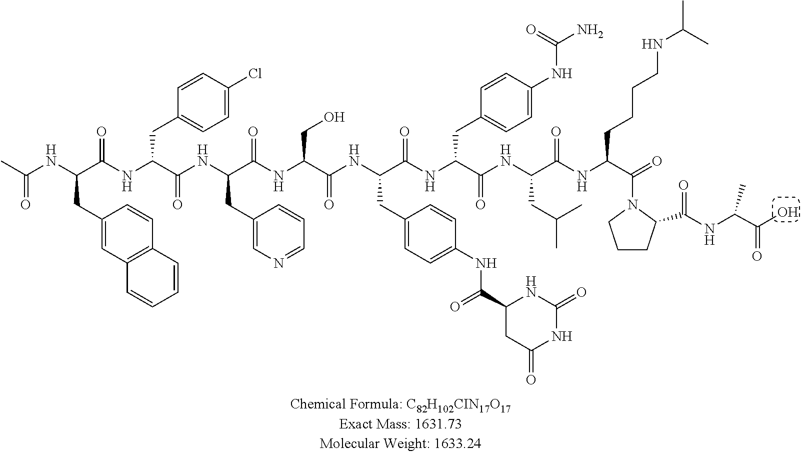 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@H](C)C(=O)O] |
| | (D-alanine, N-acetyl-3-(2-naphthalenyl)-D-alanyl-4-chloro- D-phenylalanyl-3-(3-pyridyl)-D-alanyl-L-seryl-4-[[[(4S)- hexahydro- |
| | 2,6-dioxo-4-pyrimidinyl]carbonyl]amino]-D-phenylalayl-4-[(aminocarbonyl)amino]-D- |
| | phenylalanyl-L-leucyl-N6-(1-methylethyl)-L-lysyl-L-prolyl) |
| K | 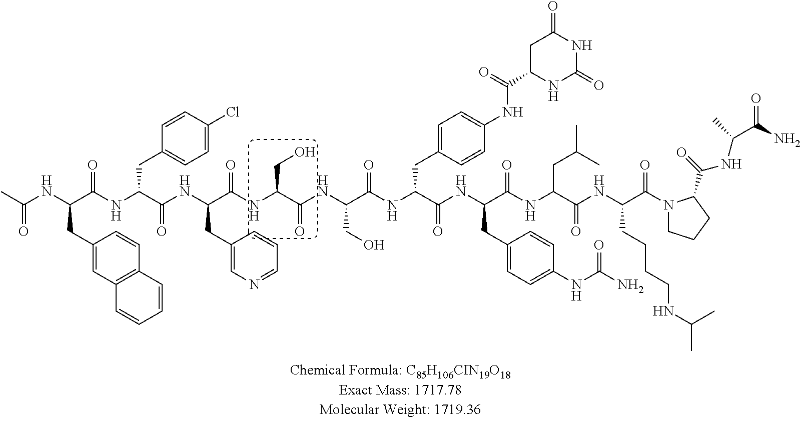 |
| | (N-Acetyl-3-(2-naphthalenyl)-D-alanyl-4-chloro-D-phenylalanyl- 3-(3-pyridyl)-D-alanyl-L-seryl-L-seryl-4-[[[(4S)-hexahydro- |
| | 2,6- dioxo-4-pyrimidinyl]carbonyl]amino]-D-phenylalanyl-4-ureido-D-phenylalanyl-L- |
| | leucyl-N6-isopropyl-L-lysyl-L-prolyl-D-alaninamide) |
| M | 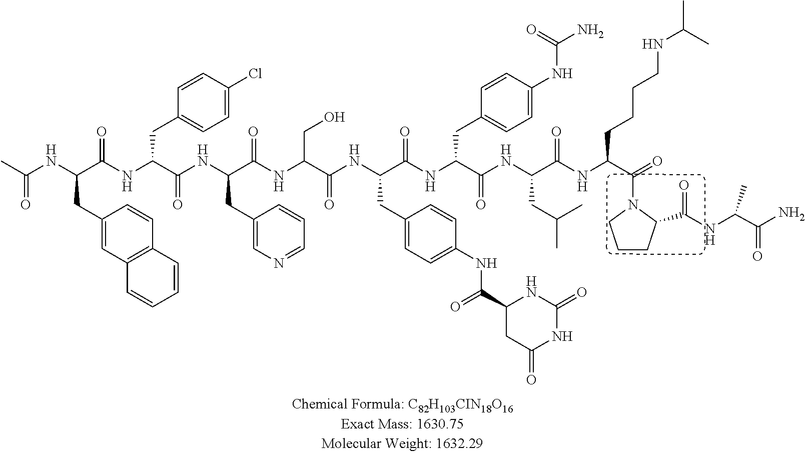 [SMILES: CC(=O)N[C@H](Cc1ccc2ccccc2c1)C(=O)N[C@H](Cc1ccc(Cl)cc1)C(=O)N[C@H](Cc1cccnc1)C(=O)NC(CO)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@H](C)C(N)=O] |
| | (D-alaninamide, N-acetyl-3-(2-naphthalenyl)-D-alanyl-4-chloro-D-phenylalanyl-3-(3-pyridyl)-D-alanyl-L-seryl-4-[[[(4S)-hexahydro- |
| | 2,6-dioxo-4-pyrimidinyl]carbonyl]amino]-D-phenylalanyl-4-[(aminocarbonyl)amino]-D- |
| | phenylalanyl-L-leucyl-N6-(1-methylethyl)-L-lysyl-D-prolyl) |

In one aspect, the invention provides a method for analyzing a sample comprising degarelix or a pharmaceutically acceptable salt thereof and at least one related impurity (e.g., one or more of compounds B, C, D, E, F, G, I, K, and M), the method comprising: (a) eluting the sample through a high pressure liquid chromatography (HPLC) column (e.g., WATERS' XSelect CSH C18 3.5 μm, 4.6×150 mm P/N 186005270) to produce a chromatogram that resolves the degarelix or pharmaceutically acceptable salt thereof and the at least one related impurity, wherein the chromatogram comprises a first peak with a first area representing degarelix in the sample, and a second peak with a second area representing the at least one related impurity in the sample; (b) determining the first area under the first peak representing the degarelix or pharmaceutically acceptable salt thereof in the sample, (c) determining the second area under the second peak representing the at least one related impurity in the sample, and (d) determining the concentration of the at least one related impurity in the sample based on the first and second areas, wherein the eluting comprises eluting the sample with a mobile phase A comprising a first aqueous solution having a first pH and a first organic solvent, and with a mobile phase B comprising a second aqueous solution having a second pH, and a second organic solvent, wherein the first and the second pH values are different. In certain embodiments, the at least one related impurity comprises compound D.

The sample analyzed may include any suitable composition containing degarelix or a pharmaceutically acceptable salt thereof and at least one related impurity (e.g., one or more of compounds B, C, D, E, F, G, I, K, and M). In some embodiments, the sample for analysis is an aqueous solution comprising degarelix or a pharmaceutically acceptable salt thereof. The aqueous solution can further comprise one or more polar organic solvents such as, for example, acetonitrile, methanol, ethanol, (iso)propanol, dimethylformamide, diethyl ether, tetrahydrofuran, ethyl acetate, or the like. Alternatively, or additionally, the aqueous solution can further comprise one or more inorganic acids (e.g., hydrochloric acid, sulfuric acid, phosphoric acid, or hydrobromic acid), organic acids (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), inorganic bases (e.g., sodium hydroxide, potassium hydroxide, calcium hydroxide, magnesium hydroxide, or ammonium hydroxide), or organic bases (e.g., methylamine, diethylamine, triethylamine, triethanolamine, ethylenediamine, tris(hydroxymethyl)methylamine, guanidine, choline, or cinchonine), or the like. In certain embodiments, the sample comprises degarelix or a pharmaceutically acceptable salt thereof, at least one related impurity (e.g., one or more of compounds B, C, D, E, F, G, I, K, and M), water, acetonitrile, and acetic acid.

The sample for analysis can have any suitable amount of the degarelix or a pharmaceutically acceptable salt thereof. For example, the sample for analysis can comprise degarelix in a concentration of from about 0.01 mg/mL to about 10 mg/mL, from about 0.1 mg/mL to about 5 mg/mL, from about 0.25 mg/mL to about 2.5 mg/mL, or from about 0.4 mg/mL to about 1 mg/mL.

In embodiments where the sample for analysis comprises acetic acid, the sample can have any suitable amount of acetic acid. For example, the sample for analysis can be applied to the column as a solution comprising acetic acid in a concentration of 0.01-1% by volume, of 0.05-0.5% by volume, or of about 0.1% by volume.

The sample for analysis can have any suitable amount of polar organic solvents. For example, the sample for analysis can be applied to the column as a solution comprising a polar solvent in a concentration of from about 1-50% by volume, from about 1-40% by volume, from about 1-30% by volume, from about 5-50% by volume, from about 5-40% by volume, from about 5-30% by volume, from about 10-50% by volume, from about 10-40% by volume, or from about 10-30% by volume. In certain embodiments, the sample for analysis comprises a polar solvent in a concentration of about 20% by volume.

The chromatogram produced according to the invention can be produced by detection of the ultraviolet (UV) absorption at any wavelength suitable for detecting degarelix. In some embodiments, the chromatogram is produced by detection of the ultraviolet (UV) absorption at about 245 nm of the degarelix and the at least one related impurity eluted from the column. The concentration of the at least one related impurity in the sample can be determined based on the first and second areas in the generated chromatogram using one or more techniques known to one skilled in the art.

The HPLC column used in accordance with the invention can be any suitable HPLC column containing a chromatographic resin for use in reverse phase chromatography. As used herein, “reverse phase chromatography” refers to any chromatographic technique that utilizes a hydrophobic stationary phase and an aqueous or hydrophilic mobile phase to analyze and/or separate sample materials. Accordingly, the chromatographic resins described herein are typically hydrophobic chromatographic resins. For example, the chromatographic resin can be octadecyl carbon chain (C18)-bonded silica, C8-bonded silica, pure silica, cyano-bonded silica, phenyl-bonded silica, activated carbon, or combinations thereof. The HPLC columns described herein can have any suitable dimensions and any suitable particle size. In certain embodiments, the HPLC column is a C18 XSelect column commercially available from WATERS' (e.g., XSelect CSH C18 3.5 μm, 4.6×150 mm P/N 186005270).

Eluting the sample through a HPLC column can occur at any suitable temperature. For example, elution can occur at a temperature of from about 10° C. to about 50° C., from about 20° C. to about 40° C., or from about 25° C. to about 35° C. In some embodiments, eluting the sample through a HPLC column can occur at a temperature of about 25° C., about 30° C., or about 35° C.

Eluting the sample through a HPLC column can occur at any suitable flow rate. For example, elution can occur at a flow rate of from about 0.1 mL/min to about 10 mL/min, from about 0.5 mL/min to about 5 mL/min, or from about 0.5 mL/min to about 2 mL/min. In some embodiments, eluting the sample through a HPLC column can occur at a flow rate of about 0.5 mL/min, about 0.6 ml/min, 0.7 mL/min, about 0.8 ml/min, 0.9 mL/min, about 1 ml/min, 1.1 mL/min, about 1.2 ml/min, 1.3 mL/min, about 1.4 ml/min, or 1.5 mL/min.

Run time for eluting the sample through a HPLC column can include any suitable length of time including run times that are commonly used in reverse phase HPLC separations. For example, the run time can be from about 5 minutes to about 5 hours, from about 15 minutes to about 4 hours, from about 30 minutes to about 2 hours, or from about 45 minutes to about 1.5 hours. In some embodiments, run time is about 30 minutes, about 45 minutes, about 1 hour, about 75 minutes, or about 90 minutes.

In some embodiments, eluting the sample through a HPLC column comprises eluting the sample with a mobile phase A comprising a first aqueous solution having a first pH and a first organic solvent, and with a mobile phase B comprising a second aqueous solution having a second pH, and a second organic solvent, wherein the first and the second pH values are different. The difference between the first and the second pH values may be at least about 0.2 pH units, at least about 0.5 pH units, at least about 1 pH unit, at least about 1.5 pH units, or at least about 2 pH units. In certain embodiments, the difference between the first and the second pH values is about 2 pH units. For example, in some embodiments, the first pH is about 5.5 and the second pH is about 3.5.

The first aqueous solution can have any suitable pH, which is different from the second pH. For example, the first pH can be from about 3 to about 9, from about 4 to about 8, from about 5 to about 7, or from about 5 to about 6. In some embodiments, the pH of the first aqueous solution is about 4.5, about 5, about 5.5, about 6, or about 6.5. For example, in some embodiments, the pH of the first aqueous solution is about 5.5.

The second aqueous solution can have any suitable pH, which is different from the first pH. For example, the second pH can be from about 1 to about 7, from about 2 to about 6, from about 3 to about 5, or from about 3 to about 4. In some embodiments, the pH of the second aqueous solution is about 2.5, about 3, about 3.5, about 4, or about 4.5. For example, in some embodiments, the pH of the second aqueous solution is about 3.5.

The first and second aqueous solutions can be buffered using any suitable buffering agent which, e.g., may be in the form of a salt. For example, the first and second aqueous solutions can each independently be buffered using a suitable salt comprising phosphate, citrate, formate, acetate, or a combination thereof. In certain embodiments, the first and second aqueous solutions each comprise a phosphate salt buffer. For example, the first and second aqueous solutions each may include a potassium phosphate.

The first and second aqueous solutions can each comprise any suitable amount of the buffering agent. For example, the first and second aqueous solutions can each independently comprise from about 1 mM to about 500 mM buffering salt (e.g., potassium phosphate), from about 1 mM to about 100 mM buffering salt (e.g., potassium phosphate), or from about 1 mM to about 50 mM buffering salt (e.g., potassium phosphate). In certain embodiments, the first and second aqueous solutions can each independently comprise about 10 mM buffering salt (e.g., potassium phosphate), about 15 mM buffering salt (e.g., potassium phosphate), about 20 mM buffering salt (e.g., potassium phosphate), about 25 mM buffering salt (e.g., potassium phosphate), about 30 mM buffering salt (e.g., potassium phosphate), about 35 mM buffering salt (e.g., potassium phosphate), or about 40 mM buffering salt (e.g., potassium phosphate).

Mobile phases A and B may comprise first and second organic solvents, respectively. The first and second organic solvents may be independently chosen from any suitable organic solvent, including organic solvents that are commonly used in HPLC separations. In some embodiments, the first and second organic solvents are at least partially miscible in water. For example, the first and second organic solvents can each independently comprise acetonitrile, methanol, ethanol, (iso)propanol, dimethylformamide, diethyl ether, tetrahydrofuran, ethyl acetate, or combinations thereof. In certain embodiments, mobile phases A and B comprise the same organic solvent. For example, in some embodiments, the first and second organic solvents of mobile phases A and B, respectively, comprise acetonitrile.

Mobile phases A and B can comprise any suitable amount of the first and second organic solvents, respectively. For example, mobile phases A and B can each independently comprise from about 1-50% by volume, from about 1-40% by volume, from about 1-30% by volume, from about 5-50% by volume, from about 5-40% by volume, from about 5-30% by volume, from about 10-50% by volume, from about 10-40% by volume, or from about 10-30% by volume of the first and second organic solvents, respectively. In some embodiments, mobile phase A comprises about 25% by volume of a first organic solvent. In other embodiments, mobile phase B comprises about 35% by volume of a first organic solvent.

In certain embodiments, mobile phase A comprises 25 mM potassium phosphate solution having a pH of 5.50 and acetonitrile in a ratio of about 75:25 (solution:acetonitrile).

In certain embodiments, mobile phase B comprises 25 mM potassium phosphate solution having a pH of 3.50 and acetonitrile in a ratio of about 65:35 (solution:acetonitrile).

In an exemplary embodiment of the invention, mobile phase A comprises 25 mM potassium phosphate solution having a pH of 5.50 and acetonitrile in a ratio of about 75:25 (solution:acetonitrile) and mobile phase B comprises 25 mM potassium phosphate solution having a pH of 3.50 and acetonitrile in a ratio of about 65:35 (solution:acetonitrile).

Eluting the sample through a HPLC column may include using a gradient of mobile phase A and mobile phase B. The gradient can transition, for example, from mobile phase A to mobile phase B and/or from mobile phase B to mobile phase A. An exemplary gradient for use in at least this aspect of the invention is as follows:

| Time (min) | Mobile phase A | Mobile phase B |
|---|---|---|
| 0 | 100 | 0 |
| 5 | 100 | 0 |
| 45 | 0 | 100 |
| 46 | 100 | 0 |
| 60 | 100 | 0 |

In some embodiments, the method of the invention further comprises selecting a batch of degarelix or a pharmaceutically acceptable salt thereof, e.g., for preparation of a pharmaceutical composition for therapeutic administration to a subject in need thereof, based upon the determination of the concentration of the at least one related impurity in a sample of the batch analyzed according to the invention. Without wishing to be bound by any particular theory, it is believed that the invention provides an improved method of analyzing, quantifying, and/or separating impurities related to and/or structurally similar to degarelix or a pharmaceutically acceptable salt thereof, thereby leading to improved quality control for batch selection and/or purification of degarelix and its salts. In certain embodiments, the invention also provides a composition of degarelix or a pharmaceutically acceptable salt thereof selected by the method described herein.

In another aspect, the invention provides a method for analyzing a sample comprising degarelix or a pharmaceutically acceptable salt thereof and compound A, the method comprising: (a) eluting the sample through a high performance liquid chromatography (HPLC) column (e.g., PHENOMENEX™ Gemini NX-C18, 4.6×150 mm, 3 μm P/N 00F-4453-E0) to produce a chromatogram that resolves the degarelix and compound A, wherein the chromatogram comprises a first peak with a first area representing degarelix in the sample, and a second peak with a second area representing compound A in the sample; (b) determining the first area under the first peak representing the degarelix or pharmaceutically acceptable salt thereof in the sample, (c) determining the second area under the second peak representing the compound A in the sample, and (d) determining the concentration of the compound A in the sample based on the first and second areas, wherein the eluting comprises isocratically eluting the sample with a mobile phase.

The sample analyzed may include any suitable composition containing degarelix or a pharmaceutically acceptable salt thereof and compound A. In some embodiments, the sample for analysis is an aqueous solution comprising degarelix or a pharmaceutically acceptable salt thereof. The aqueous solution can further comprise one or more polar organic solvents such as, for example, acetonitrile, methanol, ethanol, (iso)propanol, dimethylformamide, diethyl ether, tetrahydrofuran, ethyl acetate, or the like. Alternatively, or additionally, the aqueous solution can further comprise one or more inorganic acids (e.g., hydrochloric acid, sulfuric acid, phosphoric acid, or hydrobromic acid), organic acids (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), inorganic bases (e.g., sodium hydroxide, potassium hydroxide, calcium hydroxide, magnesium hydroxide, or ammonium hydroxide), or organic bases (e.g., methylamine, diethylamine, triethylamine, triethanolamine, ethylenediamine, tris(hydroxymethyl)methylamine, guanidine, choline, or cinchonine), or the like. In certain embodiments, the sample comprises degarelix or a pharmaceutically acceptable salt thereof, compound A, water, acetonitrile, and acetic acid.

The sample for analysis can have any suitable amount of the degarelix or a pharmaceutically acceptable salt thereof. For example, the sample for analysis can comprise degarelix in a concentration of from about 0.01 mg/mL to about 10 mg/mL, from about 0.1 mg/mL to about 5 mg/mL, from about 0.25 mg/mL to about 2.5 mg/mL, or from about 0.4 mg/mL to about 1 mg/mL.

In embodiments where the sample for analysis comprises acetic acid, the sample can include any suitable amount of acetic acid. For example, the sample for analysis can be applied to the column as a solution comprising acetic acid in a concentration of 0.01-1% by volume, of 0.05-0.5% by volume, or of about 0.1% by volume.

The sample for analysis also can include any suitable amount of polar organic solvents. For example, the sample for analysis can be applied to the column as a solution comprising a polar solvent in a concentration of from about 1-50% by volume, from about 1-40% by volume, from about 1-30% by volume, from about 5-50% by volume, from about 5-40% by volume, from about 5-30% by volume, from about 10-50% by volume, from about 10-40% by volume, or from about 10-30% by volume. In certain embodiments, the sample for analysis comprises a polar solvent in a concentration of about 20% by volume.

The chromatogram can be produced by detection of the ultraviolet (UV) absorption at any wavelength suitable for detecting degarelix. In some embodiments, the chromatogram is produced by detection of the ultraviolet (UV) absorption at about 245 nm of the degarelix and the compound A eluted from the column. The concentration of the compound A in the sample can be determined based on the first and second areas in the generated chromatogram using one or more techniques known to one skilled in the art.

The HPLC column can be any suitable HPLC column containing a chromatographic resin for use in reverse phase chromatography. For example, the chromatographic resin can be octadecyl carbon chain (C18)-bonded silica, C8-bonded silica, pure silica, cyano-bonded silica, phenyl-bonded silica, activated carbon, or combinations thereof. The HPLC columns described herein can have any suitable dimensions and any suitable particle size. In certain embodiments, the HPLC column for use in the second aspect is a Gemini NX-C18 column commercially available from PHENOMENEX™ (e.g., Gemini NX-C18, 4.6×150 mm, 3 μm P/N 00F-4453-E0).

Eluting the sample through a HPLC column can occur at any suitable temperature. For example, elution can occur at a temperature of from about 10° C. to about 50° C., from about 20° C. to about 40° C., or from about 20° C. to about 30° C. In some embodiments, eluting the sample through a HPLC column can occur at a temperature of about 20° C., about 25° C., or about 30° C.

Eluting the sample through a HPLC column can occur at any suitable flow rate. For example, elution can occur at a flow rate of from about 0.1 mL/min to about 10 mL/min, from about 0.5 mL/min to about 5 mL/min, or from about 0.5 mL/min to about 2 mL/min. In some embodiments, eluting the sample through a HPLC column can occur at a flow rate of about 0.5 mL/min, about 0.6 ml/min, 0.7 mL/min, about 0.8 ml/min, 0.9 mL/min, about 1 ml/min, 1.1 mL/min, about 1.2 ml/min, 1.3 mL/min, about 1.4 ml/min, or 1.5 mL/min.

Run time for eluting the sample through a HPLC column can be any suitable length of time including run times commonly used in reverse phase HPLC separations. For example, the run time can be from about 5 minutes to about 5 hours, from about 15 minutes to about 4 hours, from about 30 minutes to about 2 hours, or from about 45 minutes to about 1.5 hours. In some embodiments, run time is about 15 minutes, 30 minutes, about 45 minutes, or about 1 hour.

In some embodiments, eluting the sample through a HPLC column comprises isocratically eluting the sample with a mobile phase. As used herein, the term “isocratic” or “isocratic elution” refers to a process of eluting a sample using a single mobile phase, i.e., the mobile phase remains constant or unchanged.

The mobile phase may include an aqueous solution and an organic solvent.

The aqueous solution of the mobile phase can have any suitable pH. For example, the pH of the aqueous solution of the mobile phase can be from about 6 to about 13, from about 7 to about 12, from about 8 to about 11, or from about 9 to about 11. In some embodiments, the pH of the aqueous solution of the mobile phase is about 9, about 9.5, about 10, about 10.5, or about 11. In certain embodiments, the pH of the aqueous solution of the mobile phase is about 10.

The aqueous solution of the mobile phase can be buffered using any suitable buffering agent which, e.g., may be in the form of a salt. For example, the aqueous solution of the mobile phase can be buffered using a salt comprising phosphate, citrate, formate, acetate, or a combination thereof. In certain embodiments, the aqueous solution of the mobile phase comprises an acetate buffer. For example, the aqueous solution of the mobile phase may include ammonium acetate.

The aqueous solution of the mobile phase can comprise any suitable amount of the buffering agent. For example, the aqueous solution of the mobile phase can comprise from about 1 mM to about 500 mM buffering salt (e.g., ammonium acetate), from about 1 mM to about 250 mM buffering salt (e.g., ammonium acetate), or from about 1 mM to about 100 mM buffering salt (e.g., ammonium acetate). In certain embodiments, the mobile phase may include about 30 mM buffering salt (e.g., ammonium acetate), about 35 mM buffering salt (e.g., ammonium acetate), about 40 mM buffering salt (e.g., ammonium acetate), about 45 mM buffering salt (e.g., ammonium acetate), about 50 mM buffering salt (e.g., ammonium acetate), about 55 mM buffering salt (e.g., ammonium acetate), or about 60 mM buffering salt (e.g., ammonium acetate).

The mobile phase also may include an organic solvent. The organic solvent of the mobile phase may be chosen from any suitable organic solvent. In some embodiments, the organic solvent is at least partially miscible in water. For example, the organic solvent can comprise acetonitrile, methanol, ethanol, (iso)propanol, dimethylformamide, diethyl ether, tetrahydrofuran, ethyl acetate, or combinations thereof. In certain embodiments, the organic solvent of the mobile phase comprises acetonitrile.

The mobile phase can comprise any suitable amount of the organic solvent. For example, the mobile phase can comprise from about 1-50% by volume, from about 1-40% by volume, from about 1-30% by volume, from about 5-50% by volume, from about 5-40% by volume, from about 5-30% by volume, from about 10-50% by volume, from about 10-40% by volume, or from about 20-40% by volume of the organic solvent. In certain embodiments, the mobile phase comprises about 35% by volume of the organic solvent.

In some embodiments, the mobile phase comprises an ammonium acetate buffer solution and acetonitrile. In certain embodiments, the ammonium acetate buffer solution comprises from about 1 mM to about 100 mM ammonium acetate (e.g., about 30 mM, about 35 mM, about 40 mM, about 45 mM, about 50 mM, about 55 mM, or about 60 mM) and has a pH of about 9 to about 11 (e.g., about 9, about 9.5, about 10, about 10.5, or about 11). In some embodiments, the ammonium acetate buffer solution comprises about 45 mM ammonium acetate, has a pH of about 10, and the ratio of ammonium acetate buffer solution to acetonitrile in the mobile phase is about 65:35.

In some embodiments, the invention further comprises selecting a batch of degarelix or a pharmaceutically acceptable salt thereof e.g., for preparation of a pharmaceutical composition for therapeutic administration to a subject in need thereof, based upon the determination of the concentration of compound A in a sample of the batch analyzed according to the invention. Without wishing to be bound by any particular theory, it is believed that the invention provides an improved method of analyzing, quantifying and/or separating impurities that are related to and/or structurally similar to degarelix or a pharmaceutically acceptable salt thereof, which impurities are not otherwise sufficiently separable and or distinguishable from degarelix and/or from each other, thereby leading to improved quality control for batch selection. More particularly, it is believed that the invention provides an improved method to effectively separate compound A from degarelix or a salt thereof and/or to quantify compound A in a sample comprising degarelix or a salt thereof. In certain embodiments, the invention also provides a batch of degarelix or a pharmaceutically acceptable salt thereof selected according to the invention.

In a further aspect, the invention provides a method for analyzing a sample comprising degarelix or a pharmaceutically acceptable salt thereof, compound A, and at least one related impurity other than compound A, the method comprising: (a) eluting a first portion of the sample through a high performance liquid chromatography (HPLC) column (e.g., WATERS™ XSelect CSH C18 3.5 μm, 4.6×150 mm P/N 186005270) to produce a first chromatogram that resolves the degarelix or pharmaceutically acceptable salt thereof and the at least one related impurity other than compound A (e.g., compounds B, C, D, E, F, G, I, K, and M), the first chromatogram comprising a first peak with a first area representing degarelix in the sample, and a second peak with a second area representing the at least one related impurity in the sample, wherein the eluting of the first portion of sample comprises eluting with a mobile phase A comprising a first aqueous solution having a first pH and a first organic solvent, and with a mobile phase B comprising a second aqueous solution having a second pH and a second organic solvent, wherein the first and the second pH values are different, and (b) eluting a second portion of the sample through a HPLC column (e.g., PHENOMENEX™ Gemini NX-C18, 4.6×150 mm, 3 μm P/N 00F-4453-E0), to produce a second chromatogram that resolves the degarelix and compound A, the second chromatogram comprising a first peak with a first area representing degarelix in the sample, and a second peak with a second area representing compound A in the sample, wherein the eluting of the second portion of sample comprises isocratically eluting with a mobile phase, (c) determining the first areas under the first peaks representing the degarelix or pharmaceutically acceptable salt thereof in the first and second chromatograms, (d) determining the second area under the peak representing the at least one related impurity in the first chromatogram, (e) determining the second area under the second peak representing the compound A in the second chromatogram, and (f) determining the concentrations of the at least one related impurity and the compound A in the sample based on the first and second areas of the first and second chromatograms.

This aspect of the invention can combine any particular embodiments of other aspects of the invention described herein to determine the amount of and/or separate compound A and at least one related impurity other than compound A. In certain embodiments, the at least one related impurity other than compound A comprises compound D. Without wishing to be bound by any particular theory, it is believed that, due to their structural similarity (e.g., structural or electronic), compounds A and D are uniquely challenging to separate and quantify by conventional methods. However, utilizing the method of the invention that comprises eluting with mobile phases A and B as described herein allows one to quantify compound D in addition to other related impurities, and utilizing the method of the invention which comprises resolving degarelix and compound A as described herein allows one to quantify compound A. Accordingly, the present invention allows one to readily separate and/or quantify compounds A and D in a batch of degarelix or a salt thereof. Thus, the invention provides purified degarelix or a pharmaceutically acceptable salt thereof that comprises not more than 0.3 wt. % (e.g., not more than 0.2 wt. %, not more than 0.15 wt. %, not more than 0.1 wt. %, or not more than 0.01 wt. %) of compound A and not more than 0.3 wt. % (e.g., not more than 0.2 wt. %, not more than 0.15 wt. %, not more than 0.1 wt. %, or not more than 0.01 wt. %) of compound D relative to the degarelix or pharmaceutically acceptable salt thereof. In certain embodiments, the invention provides purified degarelix or a pharmaceutically acceptable salt thereof that comprises not more than 0.2 wt. % of compound A and not more than 0.15 wt. % of compound D relative to the degarelix or pharmaceutically acceptable salt thereof.

In some embodiments, the invention further comprises selecting a batch of degarelix or a pharmaceutically acceptable salt thereof, e.g., for preparation of a pharmaceutical composition for therapeutic administration to a subject in need thereof, based upon the determination of the concentration of at least one related impurity other than compound A (e.g., compound D) and compound A. Without wishing to be bound by any particular theory, the invention is believed to provide an improved method of analyzing and/or quantifying degarelix or a pharmaceutically acceptable salt thereof which contains compound A and at least one other related impurity, e.g., compound D, thereby leading to improved quality control for batch selection, particularly by providing an improved way to separate and/or quantify compound D and compound A in a batch of degarelix. In certain embodiments, the invention also provides a batch of degarelix or a pharmaceutically acceptable salt thereof selected by this method.

In some embodiments, the batch of degarelix or pharmaceutically acceptable salt thereof selected in accordance with the invention comprises not more than 0.3 wt. % (e.g., not more than 0.2 wt. %, not more than 0.15 wt. %, not more than 0.1 wt. %, or not more than 0.01 wt. %) of compound A and not more than 0.3 wt. % (e.g., not more than 0.2 wt. %, not more than 0.15 wt. %, not more than 0.1 wt. %, or not more than 0.01 wt. %) of compound D relative to degarelix or a pharmaceutically acceptable salt thereof. In certain embodiments, following storage of the batch of degarelix or pharmaceutically acceptable salt thereof selected by the method of the invention at about 2-8° C. for about 24 months, the batch comprises not more than 0.3 wt. % (e.g., not more than 0.2 wt. %, not more than 0.15 wt. %, not more than 0.1 wt. %, or not more than 0.01 wt. %) of compound A and not more than 0.3 wt. % (e.g., not more than 0.2 wt. %, not more than 0.15 wt. %, not more than 0.1 wt. %, or not more than 0.01 wt. %) of compound D relative to degarelix or a pharmaceutically acceptable salt thereof.

In yet another aspect, the invention provides a method of purifying degarelix or a pharmaceutically acceptable salt thereof which contains compound A, the method comprising: (a) eluting the sample through a chromatographic column with a mobile phase to separate the degarelix or a pharmaceutically acceptable salt thereof and compound A, thereby producing a purified comprising degarelix or a pharmaceutically acceptable salt thereof, wherein the eluting comprise isocratically eluting the sample from the chromatographic column with the mobile phase.

The chromatographic column for use in the purification method of the invention can be any suitable column containing a chromatographic resin for use in reverse phase chromatography. For example, the chromatographic resin can be octadecyl carbon chain (C18)-bonded silica, C8-bonded silica, pure silica, cyano-bonded silica, phenyl-bonded silica, activated carbon, or combinations thereof. In some embodiments, the chromatographic column is a preparatory high performance liquid chromatography (HPLC) column. In certain embodiments, the chromatographic column is an organo-silica grafted C18 resin having an average particle size of about 2 μm to about 20 μm (e.g., about 2 μm to about 10 μm or about 2 μm to about 5 μm) and a pore size of about 100 Å to about 120 Å (e.g., about 100 Å, about 110 Å, or about 120 Å).

The mobile phase can comprise an aqueous solution and an organic solvent.

The aqueous solution of the mobile phase used in the purification method of the invention can have any suitable pH. For example, the pH of the aqueous solution of the mobile phase can be from about 6 to about 13, from about 7 to about 12, from about 8 to about 11, or from about 9 to about 11. In some embodiments, the pH of the aqueous solution of the mobile phase is about 9, about 9.5, about 10, about 10.5, or about 11. In certain embodiments, the pH of the aqueous solution of the mobile phase is about 10.

The aqueous solution of the mobile phase in the purification method of the invention can be buffered using any suitable agent which, e.g., can be in the form of a salt for buffering a solution. For example, the aqueous solution of the mobile phase can be buffered using a salt comprising phosphate, citrate, formate, acetate, or a combination thereof. In certain embodiments, the aqueous solution of the mobile phase comprises an acetate buffer. In some embodiments, the aqueous solution of the mobile phase comprises ammonium acetate.

The aqueous solution of the mobile phase of the purification method of the invention can comprise any suitable amount of the buffering agent. For example, the aqueous solution of the mobile phase can comprise from about 1 mM to about 500 mM buffering salt (e.g., ammonium acetate), from about 1 mM to about 250 mM buffering salt (e.g., ammonium acetate), or from about 1 mM to about 100 mM buffering salt (e.g., ammonium acetate). In certain embodiments, the mobile phase can comprise about 30 mM buffering salt (e.g., ammonium acetate), about 35 mM buffering salt (e.g., ammonium acetate), about 40 mM buffering salt (e.g., ammonium acetate), about 45 mM buffering salt (e.g., ammonium acetate), about 50 mM buffering salt (e.g., ammonium acetate), about 55 mM buffering salt (e.g., ammonium acetate), or about 60 mM buffering salt (e.g., ammonium acetate).

The mobile phase of the purification method of the invention also may include an organic solvent. The organic solvent of the mobile phase may be chosen from any suitable organic solvent including organic solvents that are commonly used in reverse phase HPLC separations. In some embodiments, the organic solvent is at least partially miscible in water. For example, the organic solvent can comprise acetonitrile, methanol, ethanol, (iso)propanol, dimethylformamide, diethyl ether, tetrahydrofuran, ethyl acetate, or combinations thereof. In certain embodiments, the organic solvent of the mobile phase comprises acetonitrile.

The mobile phase of the purification method of the invention can comprise any suitable amount of the organic solvent. For example, the mobile phase can comprise from about 1-50% by volume, from about 1-40% by volume, from about 1-30% by volume, from about 5-50% by volume, from about 5-40% by volume, from about 5-30% by volume, from about 10-50% by volume, from about 10-40% by volume, or from about 20-40% by volume of the organic solvent. In certain embodiments, the mobile phase comprises about 35% by volume of the organic solvent.

In some embodiments, the mobile phase used in the purification method of the invention comprises an ammonium acetate buffer solution and acetonitrile. In certain embodiments, the ammonium acetate buffer solution comprises from about 1 mM to about 100 mM ammonium acetate (e.g., about 30 mM, about 35 mM, about 40 mM, about 45 mM, about 50 mM, about 55 mM, or about 60 mM) and has a pH of about 9 to about 11 (e.g., about 9, about 9.5, about 10, about 10.5, or about 11). In some embodiments, the ammonium acetate buffer solution comprises about 45 mM ammonium acetate, has a pH of about 10, and the ratio of ammonium acetate buffer solution to acetonitrile in the mobile phase is about 65:35.

The purified degarelix or a pharmaceutically acceptable salt thereof obtained by the purification method of the invention preferably comprises not more than 0.3 wt. % (e.g., not more than 0.2 wt. %, not more than 0.15 wt. %, not more than 0.1 wt. %, or not more than 0.01 wt. %) of compound A relative to the degarelix or pharmaceutically acceptable salt thereof. Accordingly, the invention also provides a purified form of degarelix or a pharmaceutically acceptable salt thereof comprising not more than 0.3 wt. % (e.g., not more than 0.2 wt. %, not more than 0.15 wt. %, not more than 0.1 wt. %, or not more than 0.01 wt. %) of compound A relative to the degarelix or pharmaceutically acceptable salt thereof. The invention further provides a pharmaceutical composition comprising an excipient and purified degarelix or a pharmaceutically acceptable salt thereof comprising not more than 1.0 wt. % (e.g., not more than 0.5 wt. %, not more than 0.3 wt. %, not more than 0.2 wt. %, or not more than 0.1 wt. %) of compound A relative to degarelix or a pharmaceutically acceptable salt thereof. In certain embodiments, following storage of the pharmaceutical composition comprising an excipient and purified degarelix or a pharmaceutically acceptable salt thereof at about 2-8° C. for about 24 months, the pharmaceutical composition comprises not more than 1.0 wt. % (e.g., not more than 0.5 wt. %, not more than 0.3 wt. %, not more than 0.2 wt. %, or not more than 0.1 wt. %) of compound A and not more than 1.0 wt. % (e.g., not more than 0.5 wt. %, not more than 0.3 wt. %, not more than 0.2 wt. %, or not more than 0.1 wt. %) of compound D relative to degarelix or a pharmaceutically acceptable salt thereof. The pharmaceutical composition of the invention preferably includes a therapeutically effective amount of the purified degarelix or pharmaceutically acceptable salt thereof produced according to the invention.

The pharmaceutical composition can be a liquid or a solid, e.g., a lyophilized solid. In some embodiments, the pharmaceutical composition further comprises one or more pharmaceutically acceptable excipients. For example, the pharmaceutical composition can comprise one or more excipients that may serve to protect the degarelix or pharmaceutically acceptable salt thereof during manufacture and/or storage, such as a sugar, amino acid, polymer, surfactant, buffer, antioxidant, or preservative. In some embodiments, the pharmaceutical composition can contain pharmaceutically acceptable auxiliary substances as required to approximate physiological conditions such as pH adjusting and buffering agents, tonicity adjusting agents, e.g., sodium acetate, sodium chloride, potassium chloride, calcium chloride, sodium lactate, mannitol and the like. The concentration of the purified degarelix or pharmaceutically acceptable salt thereof in these formulations can vary as one of ordinary skill in the art will appreciate, and may be selected based on, e.g., fluid volumes, viscosities, body weight, and the like, in accordance with the particular mode of administration selected and the patient's needs.

### EMBODIMENTS

Aspects, including embodiments, of the present subject matter described herein may be beneficial alone or in combination with one or more other aspects or embodiments. Without limiting the foregoing description, certain non-limiting embodiments of the invention are provided below. As will be apparent to those of skill in the art upon reading this disclosure, each of the individually numbered aspects or embodiments may be used or combined with any of the other individually numbered aspects disclosed herein to provide support for all such combinations of aspects, but is not limited to combinations of aspects or embodiments explicitly provided below:

1. A method for analyzing a sample comprising degarelix or a pharmaceutically acceptable salt thereof and at least one related impurity, the method comprising:
(a) eluting the sample through a high pressure liquid chromatography (HPLC) column to produce a chromatogram that resolves the degarelix or pharmaceutically acceptable salt thereof and the at least one related impurity, wherein the chromatogram comprises a first peak with a first area representing degarelix in the sample, and a second peak with a second area representing the at least one related impurity in the sample;
(b) determining the first area under the first peak representing the degarelix or pharmaceutically acceptable salt thereof in the sample,
(c) determining the second area under the second peak representing the at least one related impurity in the sample, and
(d) determining the concentration of the at least one related impurity in the sample based on the first and second areas,
   wherein the eluting comprises eluting the sample with a mobile phase A comprising a first aqueous solution having a first pH and a first organic solvent, and with a mobile phase B comprising a second aqueous solution having a second pH, and a second organic solvent, wherein the first and the second pH values are different.

2. The method of embodiment 1, wherein the difference between the first and the second pH values is at least about 1 pH unit.

3. The method of embodiment 1 or embodiment 2, wherein the first pH is about 5.5 and the second pH is about 3.5.

4. The method of any one of embodiments 1-3, wherein the first and second aqueous solutions of mobile phases A and B, respectively, comprise a phosphate buffer.

5. The method of any one of embodiments 1-4, wherein the first and second organic solvents of mobile phases A and B, respectively, comprise acetonitrile.

6. The method of any one of embodiments 1-5, wherein the mobile phase A comprises 25 mM potassium phosphate solution having a pH of 5.50 and acetonitrile in a ratio of about 75:25 (solution:acetonitrile).

7. The method of any one of embodiments 1-6, wherein the mobile phase B comprises 25 mM potassium phosphate solution having a pH of 3.50 and acetonitrile in a ratio of about 65:35 (solution:acetonitrile).

8. The method of any one of embodiments 1-7, wherein the eluting is performed using a gradient from mobile phase A to mobile phase B according to the following conditions:

| Time (min) | Mobile phase A | Mobile phase B |
|---|---|---|
| 0 | 100 | 0 |
| 5 | 100 | 0 |
| 45 | 0 | 100 |
| 46 | 100 | 0 |
| 60 | 100 | 0 |

9. The method of any one of embodiments 1-8, wherein the sample is applied to the column as a solution comprising acetic acid in a concentration of 0.01-1% by volume.

10. The method of any one of embodiments 1-9, wherein the chromatogram is produced by detection of the ultraviolet (UV) absorption at about 245 nm of the degarelix and the at least one related impurity eluted from the column.

11. The method of any one of embodiments 1-10, wherein the at least one related impurity comprises compound B, C, D, E, F, G, I, K, or M.

12. The method of any one of embodiments 1-11, wherein the sample comprises degarelix acetate.

13. The method of any one of embodiments 1-12, further comprising selecting a batch of degarelix or a pharmaceutically acceptable salt thereof for therapeutic administration to a subject in need thereof, based upon the determination of the concentration of the at least one related impurity.

14. A batch of degarelix or a pharmaceutically acceptable salt thereof selected by the method of embodiment 13.

15. A method for analyzing a sample comprising degarelix or a pharmaceutically acceptable salt thereof and compound A, the method comprising:
(a) eluting the sample through a high performance liquid chromatography (HPLC) column to produce a chromatogram that resolves the degarelix and compound A, wherein the chromatogram comprises a first peak with a first area representing degarelix in the sample, and a second peak with a second area representing compound A in the sample;
(b) determining the first area under the first peak representing the degarelix or pharmaceutically acceptable salt thereof in the sample,
(c) determining the second area under the second peak representing the compound A in the sample, and
(d) determining the concentration of the compound A in the sample based on the first and second areas, wherein the eluting comprises isocratically eluting the sample with a mobile phase.

16. The method of embodiment 15, wherein the mobile phase comprises an ammonium acetate buffer solution and acetonitrile.

17. The method of embodiment 16 or embodiment 17, wherein the ammonium acetate buffer solution comprises 45 mM ammonium acetate and has a pH of about 10.0.

18. The method of any one of embodiments 15-17, wherein the ratio of ammonium acetate buffer solution to acetonitrile in the mobile phase is about 65:35.

19. The method of any one of embodiments 15-18, wherein the sample is applied to the column as a solution comprising acetic acid in a concentration of 0.01-1% by volume.

20. The method of any one of embodiments 15-19, wherein the chromatogram is produced by detection of the ultraviolet (UV) absorption at about 245 nm of the degarelix and the compound A eluted from the column.

21. The method of any one of embodiments 15-20, wherein the sample comprises degarelix acetate.

22. The method of any one of embodiments 15-21, further comprising selecting a batch of degarelix or a pharmaceutically acceptable salt thereof for therapeutic administration to a subject in need thereof, based upon the determination of the concentration of the compound A.

23. A batch of degarelix or a pharmaceutically acceptable salt thereof selected by the method of embodiment 22.

24. A method for analyzing a sample comprising degarelix or a pharmaceutically acceptable salt thereof, compound A, and at least one related impurity other than compound A, the method comprising:
(a) eluting a first portion of the sample through a high performance liquid chromatography (HPLC) column to produce a first chromatogram that resolves the degarelix or pharmaceutically acceptable salt thereof and the at least one related impurity, the first chromatogram comprising a first peak with a first area representing degarelix in the sample, and a second peak with a second area representing the at least one related impurity in the sample, wherein the eluting of the first portion of sample comprises eluting with a mobile phase A comprising a first aqueous solution having a first pH and a first organic solvent, and with a mobile phase B comprising a second aqueous solution having a second pH and a second organic solvent, wherein the first and the second pH values are different, and
(b) eluting a second portion of the sample through a HPLC column, to produce a second chromatogram that resolves the degarelix and compound A, the second chromatogram comprising a first peak with a first area representing degarelix in the sample, and a second peak with a second area representing compound A in the sample, wherein the eluting of the second portion of sample comprises isocratically eluting with a mobile phase,
(c) determining the first areas under the first peaks representing the degarelix or pharmaceutically acceptable salt thereof in the first and second chromatograms,
(d) determining the second area under the peak representing the at least one related impurity in the first chromatogram,
(e) determining the second area under the second peak representing the compound A in the second chromatogram, and determining the concentrations of the at least one related impurity and the compound A in the sample based on the first and second areas of the first and second chromatograms.

25. The method of embodiment 24, wherein the at least one related impurity comprises compound D.

26. The method of embodiment 24 or embodiment 25, wherein the sample comprises degarelix acetate.

27. The method of any one of embodiments 24-26, further comprising selecting a batch of degarelix or a pharmaceutically acceptable salt thereof for therapeutic administration to a subject in need thereof, based upon the determination of the concentration of the at least one related impurity and the compound A.

28. The batch of degarelix or a pharmaceutically acceptable salt thereof selected by the method of embodiment 27.

29. The batch of embodiment 28, wherein following storage of the batch at about 2-8° C. for about 24 months, the batch comprises not more than 0.3 wt. % of compound A and not more than 0.3 wt. % of compound D relative to degarelix or a pharmaceutically acceptable salt thereof.

30. The batch of embodiment 28 or embodiment 29, wherein the batch comprises not more than 0.3 wt. % of compound A and not more than 0.3 wt. % of compound D relative to degarelix or a pharmaceutically acceptable salt thereof.

31. A method of purifying degarelix or a pharmaceutically acceptable salt thereof containing compound A, the method comprising:
(a) eluting the degarelix through a chromatographic column with a mobile phase to separate the degarelix or pharmaceutically acceptable salt thereof and compound A, to produce a purified form of degarelix or pharmaceutically acceptable salt thereof, and
(b) isolating the purified degarelix or pharmaceutically acceptable salt thereof, wherein the eluting comprises isocratically eluting the sample from the chromatographic column with the mobile phase.

32. The method of embodiment 31, wherein the chromatographic column is a preparatory high performance liquid chromatography (HPLC) column.

33. The method of embodiment 31 or embodiment 32, wherein the chromatographic column is an organo-silica grafted C18 resin having an average particle size of about 2 μm to about 20 μm and a pore size of about 100 Å to about 120 Å.

34. The method of any one of embodiments 31-33, wherein the mobile phase comprises an ammonium acetate buffer solution and acetonitrile.

35. The method of any one of embodiments 31-34, wherein the ammonium acetate buffer solution comprises 45 mM ammonium acetate and has a pH of about 10.0.

36. The method of any one of embodiments 31-35, wherein the ratio of ammonium acetate buffer solution to acetonitrile in the mobile phase is about 65:35.

37. The method of any one of embodiments 31-36, wherein the degarelix is applied to the chromatographic column as a solution comprising acetic acid in a concentration of 0.01-1% by volume.

38. The method of any one of embodiments 31-37, wherein the degarelix comprises degarelix acetate.

39. The method of any one of embodiments 31-38, wherein the purified degarelix or pharmaceutically acceptable salt thereof comprises not more than 0.3 wt. % of compound A relative to the degarelix or a pharmaceutically acceptable salt thereof.

40. The method of any one of embodiments 31-39, wherein the purified degarelix or pharmaceutically acceptable salt thereof comprises not more than 0.2 wt. % of compound A relative to degarelix or a pharmaceutically acceptable salt thereof.

41. The method of any one of embodiments 31-40, wherein the purified degarelix or pharmaceutically acceptable salt thereof comprises not more than 0.1 wt. % of compound A relative to degarelix or a pharmaceutically acceptable salt thereof.

42. Purified degarelix prepared according to embodiment 39.

43. Purified degarelix prepared according to embodiment 40.

44. Purified degarelix prepared according to embodiment 41.

45. A pharmaceutical composition comprising a carrier and the purified degarelix or pharmaceutically acceptable salt thereof of any one of embodiments 42-44.

46. Purified degarelix or pharmaceutically acceptable salt thereof comprising not more than 0.3 wt. % of compound A and not more than 0.3 wt. % of compound D relative to the degarelix or a pharmaceutically acceptable salt thereof.

47. The purified degarelix of embodiment 46, comprising not more than 0.2 wt. % of compound A and not more than 0.15 wt. % of compound D relative to the degarelix or a pharmaceutically acceptable salt thereof.

The following examples further illustrate the invention but, of course, should not be construed as in any way limiting its scope.

### Example 1

The following example describes a method for the analysis and/or separation of certain impurities (e.g., compound I, compound M, compound E, compound K, compound D, compound G, compound B, and/or compound C) from degarelix.

A resolution solution was prepared by accurately weighing and transferring about 27.6 mg degarelix raw material (equivalent to approximately 24 mg degarelix after correcting for moisture and acetic acid content) into a 50-mL volumetric flask. The degarelix material used should contain approximately 0.10 wt. % compound M, 0.25 wt. % to 0.35 wt. % compound E, 0.10 wt. % to 0.25 wt. % compound K, and 0.25 wt. % compound A. These impurities may be spiked into the resolution solution with appropriate amounts to achieve the proper concentrations. The resulting solution was diluted (using a 0.1% acetic acid, 20% acetonitrile in water solution) to approximately 0.48 mg/mL degarelix and approximately 0.10 wt. % compound M, 0.25 wt. % to 0.35 wt. % compound E, 0.10 wt. % to 0.25 wt. % compound K, and 0.25 wt. % compound A.

The resolution solution was analyzed by high performance liquid chromatography (HPLC) using the following parameters, and separation was carried out using a linear gradient according to Table 2:
Mobile Phase A: pH 5.5 Phosphate Buffer: ACN (75:25)
Mobile Phase B: pH 3.5 Phosphate Buffer: ACN (65:35)
Column: WATERS™ XSelect CSH C18 3.5 μm, 4.6×150 mm P/N 186005270
Column Temperature: 30.0±3.0° C.
Flow Rate: 1.0 mL/min.
Injection Volume: 10
Autosampler Temperature: 5±3° C.
Detection: UV at 245 nm
Runtime: 60 minutes
Separation Mode: Gradient

TABLE 2
Gradient Conditions

| Time (min) | Mobile phase A | Mobile phase B |
|---|---|---|
| 0 | 100 | 0 |
| 5 | 100 | 0 |
| 45 | 0 | 100 |
| 46 | 100 | 0 |
| 60 | 100 | 0 |

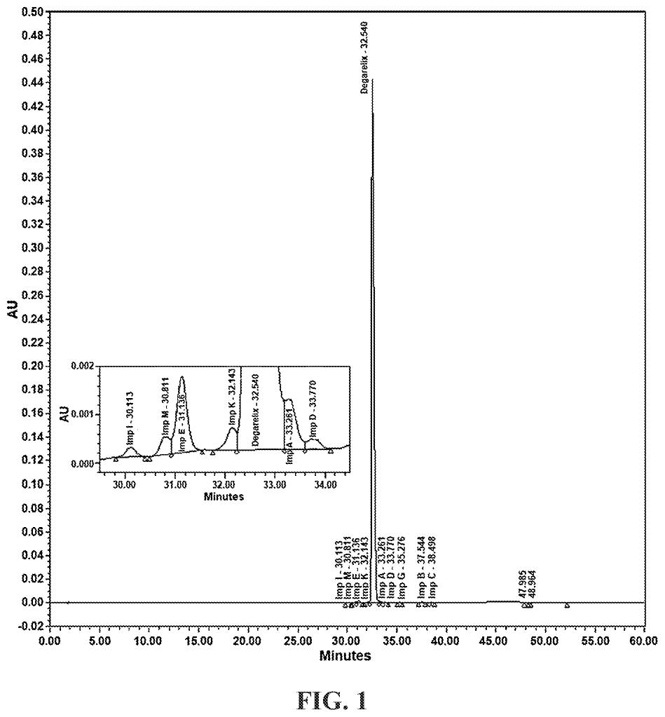

As is apparent from the results set forth in FIG. 1, the foregoing method adequately resolved compound I, compound M, compound E, compound K, compound D, compound G, compound B, and compound C from degarelix. Compound A eluted concurrently with degarelix such that the amount of degarelix could not be discerned at this stage.

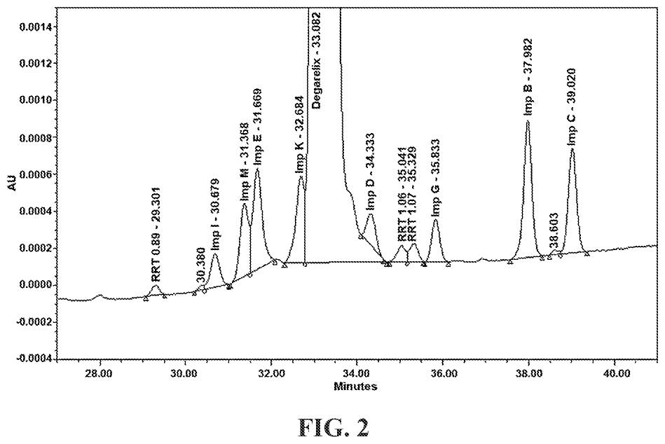

A synthetic sample of degarelix was analyzed using the foregoing HPLC parameters and the results are set forth in FIG. 2. As demonstrated by FIG. 2, the foregoing HPLC parameters adequately resolved compound I, compound M, compound E, compound K, compound D, compound G, compound B, and compound C from degarelix obtained from a synthetic sample. The relative amounts of each (Not More Than=NMT) of the impurities are set forth in Table 3.

TABLE 3
Impurity Specifications

| Compound | Amount (wt. %) |
|---|---|
| A | Not Measured |
| B | NMT 0.15 |
| C | NMT 0.15 |
| D | NMT 0.15 |
| E | NMT 0.2 |
| F | NMT 0.15 |
| G | NMT 0.15 |
| I | NMT 0.15 |
| K | NMT 0.15 |
| M | NMT 0.15 |
| All Others | NMT 0.1 |

### Example 2

The following example shows an exemplary protocol for the analysis and/or separation of compound A from degarelix.

A resolution solution was prepared by accurately weighing and transferring about 27.6 mg degarelix raw material (equivalent to approximately 24 mg degarelix after correcting for moisture and acetic acid content) into a 50-mL volumetric flask. The degarelix material used should contain approximately 0.10 wt. % compound M, 0.25 wt. % to 0.35 wt. % compound E, 0.10 wt. % to 0.25 wt. % compound K, and 0.25 wt. % compound A. These impurities may be spiked into the resolution solution with appropriate amounts to achieve the proper concentrations. The resulting solution was diluted (using a 0.1% acetic acid, 20% acetonitrile in water solution) to approximately 0.48 mg/mL degarelix and approximately 0.10 wt. % compound M, 0.25 wt. % to 0.35 wt. % compound E, 0.10 wt. % to 0.25 wt. % compound K, and 0.25 wt. % compound A.

The resolution solution was analyzed by high performance liquid chromatography (HPLC) using the following parameters, and separation was carried out using an isocratic gradient:
Mobile Phase A: 65:35
   45 mM Ammonium Acetate Buffer pH 10.0: Acetonitrile
Column: PHENOMENEX™ Gemini NX-C18, 4.6×150 mm, 3 μm P/N 00F-4453-E0
Column Temperature: 25±3° C.
Flow Rate: 0.7 mL/min.
Injection Volume: 10
Autosampler Temperature: 5±3° C.
Detection: UV at 245 nm
Runtime: 30 minutes
Separation Mode: Isocratic

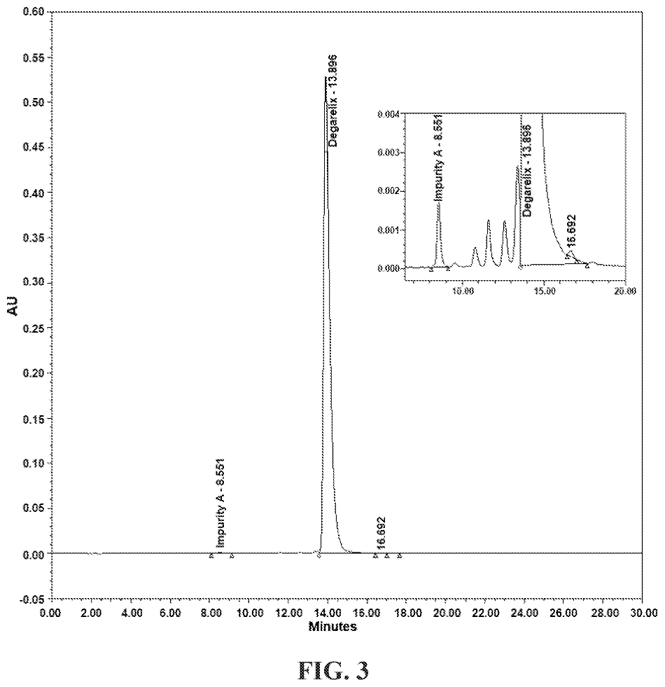

As is apparent from the results set forth in FIG. 3, the foregoing HPLC parameters adequately resolved compound A from degarelix.

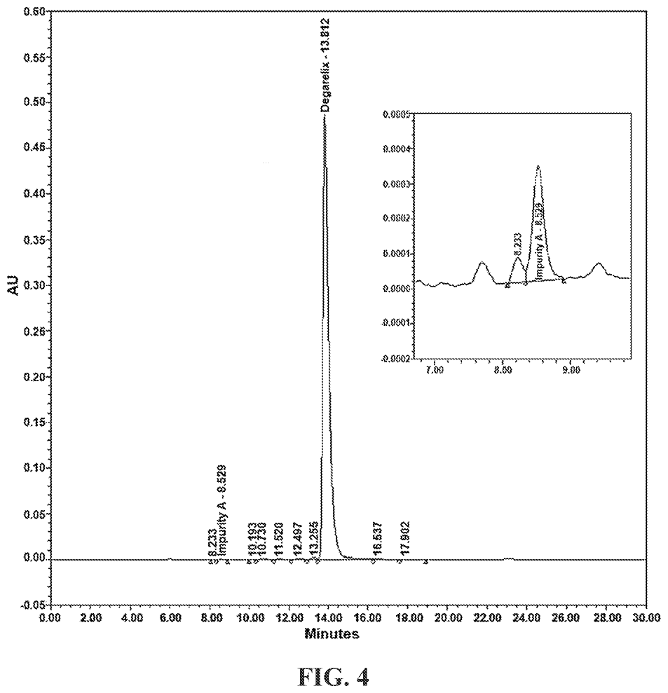

To further demonstrate the effectiveness of this method, a synthetic sample of degarelix was analyzed using the foregoing HPLC parameters and the results are set forth in FIG. 4. As demonstrated by FIG. 4, the foregoing HPLC parameters adequately resolved compound A from degarelix obtained from a synthetic sample. The relative amount of compound A was not more than (NMT) 0.2 wt. %.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of purifying degarelix or a pharmaceutically acceptable salt thereof containing compound A, the method comprising:
(a) eluting the degarelix through a chromatographic column with a mobile phase to separate the degarelix or pharmaceutically acceptable salt thereof and compound A, to produce a purified form of degarelix or pharmaceutically acceptable salt thereof, and
(b) isolating the purified degarelix or pharmaceutically acceptable salt thereof,
wherein the eluting comprises isocratically eluting the sample from the chromatographic column with the mobile phase.

2. The method of claim 1, wherein the chromatographic column is an organo-silica grafted C18 resin having an average particle size of about 2 μm to about 20 μm and a pore size of about 100 Å to about 120 Å.

3. The method of claim 1, wherein the mobile phase comprises an ammonium acetate buffer solution having a pH of about 10.0 and acetonitrile in a ratio of about 65:35 (solution:acetonitrile).

4. The method of claim 1, wherein the degarelix is applied to the chromatographic column as a solution comprising acetic acid in a concentration of 0.01-1% by volume.

5. The method of claim 1, wherein the purified degarelix or pharmaceutically acceptable salt thereof comprises not more than 0.3 wt. % of compound A relative to the degarelix or a pharmaceutically acceptable salt thereof.

6. Purified degarelix or a pharmaceutically acceptable salt thereof prepared according to claim 1.

7. The purified degarelix or pharmaceutically acceptable salt thereof according to claim 6 comprising not more than 0.3 wt. % of compound A relative to the degarelix or a pharmaceutically acceptable salt thereof.

8. A pharmaceutical composition comprising the purified degarelix or pharmaceutically acceptable salt thereof of claim 6.

9. A method for analyzing a sample comprising degarelix or a pharmaceutically acceptable salt thereof and at least one related impurity, the method comprising:
(a) eluting the sample through a high pressure liquid chromatography (HPLC) column to produce a chromatogram that resolves the degarelix or pharmaceutically acceptable salt thereof and the at least one related impurity, wherein the chromatogram comprises a first peak with a first area representing degarelix in the sample, and a second peak with a second area representing the at least one related impurity in the sample;
(b) determining the first area under the first peak representing the degarelix or pharmaceutically acceptable salt thereof in the sample,
(c) determining the second area under the second peak representing the at least one related impurity in the sample, and
(d) determining the concentration of the at least one related impurity in the sample based on the first and second areas,
wherein the eluting comprises eluting the sample with a mobile phase A comprising a first aqueous solution having a first pH and a first organic solvent, and with a mobile phase B comprising a second aqueous solution having a second pH, and a second organic solvent, wherein the first and the second pH values are different.

10. The method of claim 9, wherein the difference between the first and the second pH values is at least about 1 pH unit.

11. The method of claim 9, wherein the mobile phase A comprises a potassium phosphate solution having a pH of 5.50 and acetonitrile in a ratio of about 75:25 (solution:acetonitrile) and the mobile phase B comprises a potassium phosphate solution having a pH of 3.50 and acetonitrile in a ratio of about 65:35 (solution:acetonitrile).

12. The method of claim 9, wherein the sample is applied to the column as a solution comprising acetic acid in a concentration of 0.01-1% by volume.

13. The method of claim 9, wherein the at least one related impurity comprises compound B, C, D, E, F, G, I, K, or M.

14. A method for analyzing a sample comprising degarelix or a pharmaceutically acceptable salt thereof containing compound A and at least one related impurity other than compound A, the method comprising:
(a) eluting a first portion of the sample through a high performance liquid chromatography (HPLC) column to produce a first chromatogram that resolves the degarelix or pharmaceutically acceptable salt thereof and the at least one related impurity, the first chromatogram comprising a first peak with a first area representing degarelix in the sample, and a second peak with a second area representing the at least one related impurity in the sample, wherein the eluting of the first portion of sample comprises eluting with a mobile phase A comprising a first aqueous solution having a first pH and a first organic solvent, and with a mobile phase B comprising a second aqueous solution having a second pH and a second organic solvent, wherein the first and the second pH values are different, and
(b) eluting a second portion of the sample through a HPLC column, to produce a second chromatogram that resolves the degarelix and compound A, the second chromatogram comprising a first peak with a first area representing degarelix in the sample, and a second peak with a second area representing compound A in the sample, wherein the eluting of the second portion of sample comprises isocratically eluting with a mobile phase,
(c) determining the first areas under the first peaks representing the degarelix or pharmaceutically acceptable salt thereof in the first and second chromatograms,
(d) determining the second area under the peak representing the at least one related impurity in the first chromatogram,
(e) determining the second area under the second peak representing the compound A in the second chromatogram, and
(f) determining the concentrations of the at least one related impurity and the compound A in the sample based on the first and second areas of the first and second chromatograms.

15. The method of claim 14, wherein the at least one related impurity comprises compound D.

16. The method of claim 14, further comprising selecting a batch of degarelix or a pharmaceutically acceptable salt thereof for therapeutic administration to a subject in need thereof, based upon the determination of the concentration of the at least one related impurity and the compound A.

17. The batch of degarelix or a pharmaceutically acceptable salt thereof selected by the method of claim 16.

18. The batch of claim 17, wherein the batch comprises not more than 0.3 wt. % of compound A and not more than 0.3 wt. % of compound D relative to degarelix or a pharmaceutically acceptable salt thereof.

19. The batch of claim 17, wherein following storage of the batch at about 2-8° C. for about 24 months, the batch comprises not more than 0.3 wt. % of compound A and not more than 0.3 wt. % of compound D relative to degarelix or a pharmaceutically acceptable salt thereof.

20. A pharmaceutical composition comprising a portion of the batch of degarelix or pharmaceutically acceptable salt thereof of claim 17.
